(19) United States
(12) Patent Application Publication
(10) Pub. No.: US 2026/0148826 A1
(43) Pub. Date: May 28, 2026

(54) METHOD FOR TITRATING A MEDICAMENT

(71) Applicant: Roche Diabetes Care, Inc., Indianapolis, IN (US)

(72) Inventors: Tim Schwirtlich, Mannheim (DE); Marina Rodenas, Meylan (FR)

(21) Appl. No.: 19/453,758

(22) Filed: Jan. 20, 2026

Related U.S. Application Data
(63) Continuation of application No. PCT/US2023/070698, filed on Jul. 21, 2023.

(51) Int. Cl. G16H 20/17; G16H 10/60; G16H 50/70

(52) U.S. Cl. CPC G16H 20/17; G16H 10/60; G16H 50/70

(57) ABSTRACT

A method of titrating an antidiabetic for a patient is disclosed, along with a system for performing the method. The method includes providing a database having anonymized personal parameters for a plurality of previously managed subjects and generating cohorts from the plurality of previously managed subjects based on similarities in the anonymized personal parameters. The method further includes receiving patient specific personal parameters and identifying from the generated cohorts a patient similar cohort corresponding to the patient specific personal parameters. A titration output parameter to optimize may either be entered or computed. Based on the titration output parameter to optimize and the patient similar cohort, a customized titration protocol for the patient can then be derived.

### RELATED APPLICATIONS

This application claims priority from application PCT/US2023/070698, filed Jul. 21, 2023, the entire disclosure of which is incorporated herein by reference.

### TECHNICAL FIELD

The teachings of this disclosure generally relate to a system and a method for titrating a medicament. In particular, this disclosure relates to a system and a method for titrating an antidiabetic.

### BACKGROUND

In general, titration is the process of adjusting the dose of a medication to achieve optimal therapeutic benefit with minimal adverse effects. Titration is particularly important for medications having a narrow therapeutic index because the difference between a therapeutic dose and a dose that may cause significant side effects is comparatively small. Antidiabetics, including insulin and biosimilar insulins, are among the medications that commonly require titration to achieve proper glycemic control without causing hypoglycemic events.

Existing antidiabetic titration systems have largely pre-set titration schedules that may be based on drug manufacturer guidelines and/or basic characteristics of therapy (i.e., varying levels of settings corresponding to titration aggressiveness), but these systems do not take into account the characteristics of the patient. Alternatively, titration systems can be customized manually by the healthcare provider before the protocol is initiated, such that the starting dose, total dose, dose step adjustments, time to adjustments, and target ranges can be customized before titration begins. Whatever the selected titration protocol, if it fails, the healthcare provider adjusts the parameter settings and starts over with another titration protocol.

A typical procedure for titrating an antidiabetic (10) is shown in FIG. 1. The steps include:
a) Diagnosing a need for antidiabetic administration (12).
b) Prescribing a particular antidiabetic (14).
c) Obtaining generic titration instructions for the prescribed antidiabetic (16).
d) Establishing an administration regimen based on the generic instructions (18).
e) Teaching the patient the administration regimen (20).
f) Following the administration regimen (22).
g) Evaluating patient results (24).
h) If the titration is successful, the administration regimen is continued (28).
i) If the titration is unsuccessful, the healthcare provider manually adjusts the administration regimen (30).

In practice, such generic titration procedures are suboptimal. This is because patients starting on any particular antidiabetic, such as insulin, may respond to the treatment differently based on a number of patient-specific factors, which the generic titration procedures fail to take into account. The generic titration protocol is medication-specific, not patient-specific. The healthcare provider rarely has the tools to determine a patient's likely response and to adjust either the selected antidiabetic or the generic titration parameters to something more appropriate for the particular patient. While the healthcare provider may be able to adjust certain parameters once the titration protocol has been in place for some time, this process adds a cognitive burden for the provider and, in many cases, adjustments are made by a mere educated guess.

Often, existing approaches to titration do not result in a protocol that is truly tailored to the patient, thus reducing the likelihood of successful and timely glycemic control. Delays caused by inadequate titration protocols cause delays in needed therapy optimization for the patient, which in turn increases the risk of chronic, comorbid disease progression and/or adverse side effects. Therefore, improved tools are needed for creating individualized, reliable titration protocols.

### SUMMARY

A method is disclosed for customizing a titration protocol based upon previously successful titration protocols in patients with similar demographic and health profiles.

As used in the following, the terms “have,” “comprise” or “include” or any arbitrary grammatical variations thereof are generally open-ended terms. Thus, these terms may both refer to a situation in which, besides the feature introduced by these terms, no further features happen to be present in the entity described in this context and to a situation in which one or more further features are present. As an example, the expressions “A has B,” “A comprises B” and “A includes B” may both refer to a situation in which, besides B, no other element is present in A (i.e., a situation in which A solely and exclusively consists of B) and to a situation in which, besides B, one or more further elements are present in entity A, such as element C, elements C and D or even further elements.

Further, it shall be noted that the terms “at least one,” “one or more” or similar expressions indicating that a feature or element may be present once or more than once typically will be used only once, if at all, when introducing the respective feature or element. In the following, in most cases, when referring to the respective feature or element, the expressions “at least one” or “one or more” will not be repeated, notwithstanding the fact that the respective feature or element may be present once or more than once. It shall also be understood for purposes of this disclosure and appended claims that, regardless of whether the phrases “one or more” or “at least one” precede an element or feature appearing in this disclosure or claims, such element or feature shall not receive a singular interpretation unless it is made explicit herein. By way of non-limiting example, the terms “antidiabetic,” “personal parameter,” and “titration protocol parameter,” to name just a few, should be interpreted wherever they appear in this disclosure and claims to mean “at least one” or “one or more” regardless of whether they are introduced with the expressions “at least one” or “one or more.” All other terms used herein should be similarly interpreted unless it is made explicit that a singular interpretation is intended.

Further, as used in the following, the terms “preferably,” “more preferably,” “particularly,” “more particularly,” “specifically,” “more specifically” or similar terms are used in conjunction with optional features, without restricting alternative possibilities. Thus, features introduced by these terms are optional features and are not intended to restrict the scope of the claims in any way. The invention may, as the skilled person will recognize, be performed by using alternative features. Similarly, features introduced by “in an embodiment of the invention” or similar expressions are intended to be optional features, without any restriction regarding alternative embodiments of the invention, without any restrictions regarding the scope of the invention and without any restriction regarding the possibility of combining the features introduced in such way with other optional or non-optional features of the invention.

The terms “patient” and “subject” may be used interchangeably herein. Both refer to a person with diabetes or a person with pre-diabetes.

### Terms

The term “titration” as used herein is a broad term and is to be given its ordinary and customary meaning to a person of ordinary skill in the art and is not to be limited to a special or customized meaning. The term “titration” specifically may refer, without limitation, to a procedure, system or method used to adjust the dose and/or timing of a particular medication to achieve therapeutic effect in a patient while minimizing the adverse effects of the medication on the patient.

The term “antidiabetics” as used herein is a broad term and is to be given its ordinary and customary meaning to a person of ordinary skill in the art and is not to be limited to a special or customized meaning. The term “antidiabetics” specifically may refer, without limitation, to insulin as defined below, as well as, for example, amylinomimetic injectables, alpha-glucosidase inhibitors, biguanides, dopamine-2 agonists, dipeptidyl peptidase-4 (DPP-4) inhibitors, glucagon-like peptide-1 receptor agonists (GLP-1 receptor agonists), meglitinides, sodium-glucose transporter (SGLT) 2 inhibitors, sulfonylureas, thiazolidinediones, and other medications with similar therapeutic effects.

The term “insulin” as used herein is a broad term and is to be given its ordinary and customary meaning to a person of ordinary skill in the art and is not to be limited to a special or customized meaning. The term “insulin” specifically may refer, without limitation, to naturally occurring human or animal insulin, partially or wholly biosynthetic insulins such as biosimilar insulins, long-acting insulin, fast-acting insulin, or the like. Insulin may be delivered orally, by inhalation or by injection.

The term “personal parameter” as used herein is a broad term and is to be given its ordinary and customary meaning to a person of ordinary skill in the art and is not to be limited to a special or customized meaning. The term “personal parameter” specifically may refer, without limitation, to parameters that describe the health, demographic or other attributes of a particular person, subject or patient.

The term “health parameter” as used herein is a broad term and is to be given its ordinary and customary meaning to a person of ordinary skill in the art and is not to be limited to a special or customized meaning. The term “health parameter” specifically may refer, without limitation, to HbA1c, comorbid conditions, age, height, weight, body mass index, other medications, hypoglycemia risk level, blood glucose values, vital signs, etc.

The term “demographic parameter” as used herein is a broad term and is to be given its ordinary and customary meaning to a person of ordinary skill in the art and is not to be limited to a special or customized meaning. The term “demographic parameter” specifically may refer, without limitation, to ethnicity, age, gender, socioeconomic status, preferred modes of communication, etc.

The term “digital twin” as used herein is a broad term and is to be given its ordinary and customary meaning to a person of ordinary skill in the art and is not to be limited to a special or customized meaning. The term “digital twin” specifically may refer, without limitation, to a digital representation of a patient that serves as a digital counterpart to the patient for purposes of statistical analysis or the like. A digital twin need not be precisely identical for the purposes of this disclosure. A digital twin may be individually created using continuous glucose monitor data, activity data from, e.g., smart devices, electronic medical records or the like. Alternatively, the twin may be selected from an existing set of common profiles. The common profile that most closely parallels the patient's data can be selected and used as the digital twin.

The term “cohort” as used herein is a broad term and is to be given its ordinary and customary meaning to a person of ordinary skill in the art and is not to be limited to a special or customized meaning. The term “cohort” specifically may refer, without limitation, to a group of people who share one or more common characteristics of interest (also referred to herein as “commonalities” or “similarities”). More specifically, “cohort” may refer to a group of patients sharing one or more personal parameters, such as health and/or demographic parameters.

The term “patient similar cohort” as used herein is a broad term and is to be given its ordinary and customary meaning to a person of ordinary skill in the art and is not to be limited to a special or customized meaning. The term “patient similar cohort” specifically may refer, without limitation, to the cohort sharing common characteristics of interest with the patient for whom the custom titration protocol is being developed.

The term “titration protocol” as used herein is a broad term and is to be given its ordinary and customary meaning to a person of ordinary skill in the art and is not to be limited to a special or customized meaning. The term “titration protocol” specifically may refer, without limitation, to the procedure used to titrate a medication, including the various input parameters such as selection of an antidiabetic, starting dose, total dose, dose step adjustments, time to adjustments, target ranges, etc.

The term “titration protocol parameter” as used herein is a broad term and is to be given its ordinary and customary meaning to a person of ordinary skill in the art and is not to be limited to a special or customized meaning. The term “titration protocol parameter” specifically may refer, without limitation, to various input parameters such as selection of the antidiabetic, starting dose, total dose, dose step adjustments, time to adjustments, target ranges, etc.

The term “titration output parameter” as used herein is a broad term and is to be given its ordinary and customary meaning to a person of ordinary skill in the art and is not to be limited to a special or customized meaning. The term “titration output parameter” specifically may refer, without limitation, to parameters resulting from performing the titration protocol such as, e.g., time to achieve glycemic control, number of hypoglycemic events, number of hyperglycemic events, HbA1c, fasting blood glucose level, the percentage of time within a target blood glucose range during the titration period, etc.

The term “successful titration” as used herein is a broad term and is to be given its ordinary and customary meaning to a person of ordinary skill in the art and is not to be limited to a special or customized meaning. The term “successful titration” specifically may refer, without limitation, to a titration meeting the titration output parameter goals or threshold levels.

The term “unsuccessful titration” as used herein is a broad term and is to be given its ordinary and customary meaning to a person of ordinary skill in the art and is not to be limited to a special or customized meaning. The term “unsuccessful titration” specifically may refer, without limitation, to a titration that does not meet the titration output parameter goals or threshold levels.

The term “delivery medium” as used herein is a broad term and is to be given its ordinary and customary meaning to a person of ordinary skill in the art and is not to be limited to a special or customized meaning. The term “delivery medium” specifically may refer, without limitation, to various devices or modes that may be used alone or in combination to convey custom titration protocol instructions to the patient. For example, “delivery medium” may refer to SMS, lightweight titration service app, integration in other app (e.g., mySugr), voice-skill Alexa, Siri, Cortana, Google Home (smartphone vs. home device), bot or assistant calling the patient, personal computers, enterprise computers, dumb terminals, television screens, bot assistants, network communication devices, tablets, smart phones, smart watches and the like.

### Method

The method steps disclosed herein can be carried out in the illustrated sequence. However, alternative sequences are also possible. Further, individual or multiple method steps can be carried out in parallel, simultaneously, or repeatedly, either on their own or in groups. For example, the steps for determining titration protocol parameters need not be carried out in the precise order described below. Furthermore, the method can comprise additional method steps that are not illustrated. Independently of the fact that the term method step is used, the term “step” says nothing about the duration of the method steps. Thus, the specified method steps can, individually or in groups, be carried out briefly, but can also be carried out over a longer time period, for example, over time intervals of a number of minutes, hours, days, weeks or even months, for example, continuously or repeatedly.

The present disclosure relates to a method of customizing a titration protocol including the steps of:
a) providing a database having anonymized personal parameters for a plurality of previously managed subjects;
b) generating cohorts from the plurality of previously managed subjects based on commonalities in the anonymized personal parameters;
c) identifying successful and unsuccessful titrations within each cohort;
d) receiving patient specific personal parameters;
e) identifying from the generated cohorts a patient similar cohort corresponding to the patient specific personal parameters;
f) receiving a titration output parameter to optimize;
g) determining which titration protocol parameters contributed to success for the successful titrations and which titration protocol parameters contributed to failure for the unsuccessful titrations; and
h) based on the titration output parameter and the patient similar cohort, deriving a customized titration protocol for the patient.

In some embodiments, the method may also include the step of diagnosing a need for antidiabetic administration before performing step (a).

In some embodiments, the method may further include:
i) delivering the customized titration protocol to the patient
j) following the titration protocol
k) evaluating results of the titration
l) when the titration is successful, maintaining the administration regimen
m) when the titration is not successful, adjusting the administration regimen

### Diagnosis:

A health care provider may typically diagnose the need for antidiabetic administration. The diagnosis may occur based on periodic blood work or during a standard office visit during which fasting blood glucose is tested. The diagnosis may also occur as a result of a patient visit due to symptoms, such as frequent urination, thirst, fatigue, unexplained weight loss, cuts or wounds that heal slowly, blurred vision, etc. The diagnosis may also result, for example, from an oral glucose tolerance test.

### Identifying a Cohort for the Patient:

The titration system may include anonymized personal parameters, such as, for example, health and demographic parameters, for previously managed subjects. The previously managed subjects may be grouped into cohorts based on a similarity analysis, which may be performed using any one or a combination of techniques to determine statistical or learned similarity of data sets. Examples of techniques suitable for use in the disclosed method include, but are not limited to:

Cosine Similarity: Similarity between a patient and a database of previously managed subjects can be determined by modeling previously managed subjects as vectors of defined health and demographic parametric data points and comparing vectors using similarity measures. Vectors are feature embeddings composed of binary or numeric features representing health and demographic parametric data points such as but not limited to existing conditions, admitted medications, vital signs, lab observations and temporal relationships of those data points and clinical events.

Knowledge Graph Databases/Algorithms: Similarity between a patient and a database of previously managed subjects can be determined using knowledge graph databases. Health and demographic parameters can be vertices in the knowledge graph. For example, age, HbA1c, comorbidities, medications and body mass index may form vertices in a knowledge graph. Edges can be defined accordingly, e.g., a patient having diabetes and taking Metformin would have edges to the diabetes and Metformin vertex. Similarity between patients may be determined using, for example, metric similarity or vertex similarity. Metric similarity may be determined based on a normalized distance metric such as Euclidean distance, Manhattan distance, Levenshtein distance, Mahalanobis distance, Minkowski distance, Hamming distance, etc. Vertex similarity may be determined using, for example, neighborhood count, neighborhood selectivity, neighborhood rarity, SimRank, etc.

Artificial Intelligence/Machine Learning Modeling: Alternatively, similarity between one or more patients and a database of previously managed subjects can be determined using a machine-learning based AI model. Non-limiting examples of such learning algorithms include: K-nearest neighbor, support vector machines, naive bayes, decision trees such as random forest, logistic regression such as multinominal logistic regression, neuronal network, decision trees and Bayes network. Exemplary methods are described, below (Sammut et al., Encyclopedia of Machine Learning, 1st. Springer Publishing Company, Incorporated, 2011). Table 1 summarizes advantages and disadvantages of the methods.

Clustering: Clustering uses one or more analytical techniques for grouping a set of objects in such a way that objects in the same group are more similar to each other than to those in other groups.

K-nearest Neighbor: This is one example of a clustering method. Other deep clustering methods may also be used with this disclosure. The goal of this method is to place an object into a class with similar objects. The class for a particular object is determined based on which class appears most frequently for objects with similar parametric values. In order to determine the proximity of the objects, a similarity measure, such as, for example, the Euclidian distance is used. This method is very well suited for significantly larger data quantities.

Support Vector Machines: In this method, a hyper plane is calculated, which classifies objects into classes. For calculating the hyper plane, the distance around the class boundaries is to be maximized, which is why the Support Vector Machine is one of the ‘Large Margin Classifiers.’ An important assumption of this method is the linear separability of the data, which, however, can be expanded to higher dimensional vector spaces by means of the Kernel trick. Large data quantities are required for a classification with less overfitting.

Naive Bayes: The naive assumption is that the present variables are statistically independent from one another. This assumption is not true for most cases. In many cases, Naive Bayes nonetheless reaches a high rate of correct classification even if the attributes correlate slightly. Naive Bayes analysis is relatively simple to perform.

Regression: In a regression analysis, the relationships between a dependent variable and one or more independent variables are determined using a statistical process.

Logistic Regression: This is one example of a regression method. Other regression methods may also be used with this disclosure. In a logistic regression, the likelihood that values of a dependent variable can be attributed to values of independent variables is calculated.

Deep Learning: Deep learning is part of the broader family of machine learning methods, which is based on artificial neural networks with representation learning. The term “deep” refers to the use of multiple layers in the network. Methods of deep learning can be either supervised, semi-supervised or unsupervised. Deep learning architectures may include, but are not limited to deep neural networks, deep belief networks, deep reinforcement learning, recurrent neural networks, convolutional neural networks and transformers, etc.

Neuronal Networks: This is one example of a deep learning method. Other deep learning methods may also be used with this disclosure. Artificial neuronal networks are based on the biological structure of neurons in the brain. A simple neuronal network consists of neurons arranged in three layers. These layers are the input layer, the hidden layer and the output layer. Between the layers, all neurons are connected to one another via weights, which are optimized during a training phase.

Decision Trees: Decision trees are sorted, layered trees, which are characterized by their simple and easily comprehensible appearance. Nodes which are located close to the root are more significant for the classification of an object than nodes located close to the leaf. Decision trees often experience problems caused by overfitting. Consequently, the random forest methodology can be useful. A random forest consists of a plurality of decision trees, whereby each tree represents a subset of variables.

Bayes Networks: A Bayes network is a directed graph, which illustrates multi-variable likelihood distributions. The nodes of the network correspond to random variables and the edges show the relationships between them. For developing a Bayes network, it is helpful to describe the dependencies between the variables in as much detail as possible.

TABLE 1
Summary of advantages and disadvantages of AI/machine learning methods

| Method | Advantage | Disadvantage |
|---|---|---|
| K-nearest | Learning phase is practically non- | Finding nearest neighbor makes |
| Neighbor | existent as all training data is only | classification phase very complex and |
| | temporarily stored and only | slow for large quantities of data. |
| | evaluated when there are new | |
| | objects to classify (‘lazy learning’). | |
| Support | Special variables allow for falsely | Large quantities of data are needed for |
| Vector | assigning single data points, | a classification with as little over- |
| Machines | avoiding over-fitting. | fitting as possible |
| Naive Bayes | Reaches high accuracy and a speed | Data must be normally distributed, |
| | comparable to Decision Tree | otherwise, model is not precise. |
| | methods and Neuronal Networks | |
| | when applied to large quantities of | |
| | data. | |
| | Training time is linear with respect | |
| | to quantity of data and number of | |
| | attributes. | |
| Logistic | For classification, non-relevant | Modeling may be more difficult when |
| Regression | variables may be identified easily | many interrelationships exist between |
| | using Backwards-Elimination. | variables. |
| Decision | Decision Trees may easily be | Variance is often large. Therefore, trees |
| Trees | transformed into interpretable | should be trimmed. |
| | decision rules, following all paths | |
| | from root to leaf nodes. | |
| | Variables that occur close to the | |
| | root node due to high relevancy for | |
| | classification allow a prioritization | |
| | of the variables. | |
| Neuronal | Neuronal Networks can illustrate | A high number of hyper parameters |
| Networks | very complex problems over a large | exists, that need to be set based on |
| | range of parameters in the form of | experience for the optimization of such |
| | weight matrices. | Networks. |
| | | The training phase is very long when |
| | | the number of variables is high. |
| Bayes | A Bayes Network may be displayed | Probabilities for parameters have to be |
| Networks | in the form of a graph. | estimated, necessitating experts. |
| | | Distribution of random variables may |
| | | be difficult for more complex data, as |
| | | e.g., child nodes may follow a |
| | | Bernoulli distribution while parent |
| | | nodes follow a Gaussian distribution. |
| Regression | Allows assessment of the strength | Modeling may be more difficult when |
| | of the relationship between different | many interrelationships exist between |
| | variables. (See also Logistic | variables. |
| | Regression) | |
| Clustering | Works with multiple data types, | The choice of clustering algorithm may |
| | e.g., discrete, categorical, binary. | affect results. |
| | Can be used to identify obscure | |
| | patterns and relationships. (See | |
| | also K-nearest neighbor) | |
| Deep | Can handle large, complex data sets | Large amounts of data required for |
| Learning | as well as non-linear relationships | training. |
| | and unstructured data, missing data, | |
| | etc. (See also Neuronal networks) | |

### Data Capture:

The personal parameters, e.g., health and demographic parameters for the patient may be received into the titration system. For example, the parameters may be obtained by the titration system from an electronic medical records system or a healthcare provider may enter the patient's personal parameters using a healthcare provider interface. Non-limiting examples of healthcare provider interfaces include personal computers, enterprise computers, dumb terminals, network communication devices, tablets, smart phones, smart watches and the like. Non-limiting examples of health parameters include HbA1c, comorbid conditions, age, height, weight, body mass index, other medications, etc. Non-limiting examples of demographic parameters include ethnicity, age, gender, socioeconomic status, preferred modes of communication, etc.

The patient's personal parameters may be used to create a digital twin for the patient. The patient may then be placed into a cohort of previously managed subjects by analyzing the digital twin and the previously managed subject cohorts using similarity analysis techniques such as those discussed above. Alternatively, the patient may be placed into a cohort by using similarity analysis techniques to identify a digital twin from a set of pre-existing digital profiles. The patient may then be assigned to a cohort according to the selected digital twin.

### Customizing the Antidiabetic Type and the Titration Protocol Based on the Patient Cohort and a Selected Titration Parameter or Parameters:

A health care provider may enter one or more titration output parameters to optimize using the healthcare provider interface. Non-limiting examples of healthcare provider interfaces include personal computers, enterprise computers, dumb terminals, network communication devices, tablets, smart phones, smart watches and the like. Non-limiting examples of titration output parameters to optimize may include shortest time to achieve the target range; maximum number of patients within an expected range within a predefined time period; maximum number patients who achieved the target range on the first titration cycle; highest percentage of time in range; minimal number of hypoglycemic events, etc.

Data from previous patients with titrations satisfying the titration optimization goals can be isolated and correlative protocol parameters may be determined. At the same time, data from previous patients with titrations that were not successful according to the optimization goals can also be isolated and the correlative protocol parameters leading to failure may also be determined.

For example, each of the titration protocol parameters could be assessed in terms of their statistical correlation to the desired/undesired titration outcomes vs. all possible outcomes. This analysis may be done for the entire patient population pool, the similar patient cohort pool, or some combination thereof. Within acceptable confidence and significance levels, titration parameters at or above a minimum statistical correlation threshold (e.g., 0.7, 0.8, 0.9 or 0.95) could be considered for adjustment in the custom titration protocol.

Alternatively, a regression and clustering analysis to compare the parameters of a successful titration could be used to identify which characteristics were deterministic of success. At the same time, a regression and clustering analysis to compare the parameters of an unsuccessful titration could be used to identify which characteristics were deterministic of failure. A logistic regression approach may include, for example:
1. Collecting patient personal parameters, including a success variable, which may be, e.g., binary.
2. Using the success variable as the response/target variable.
3. Initially assuming all titration protocol parameters are predictors for the regression.
4. Identifying titration protocol parameters with a significant p-value, meaning that such parameters have a significant effect on the outcome and are to some extent deterministic of success.
5. Carrying titration protocol parameters identified in step (4) as meeting the significance criteria to the next step for analysis with the patient similar cohort.

To determine which parameters to adjust to form a custom titration protocol, the significant/correlative parameters can be assessed against their respective protocol outcomes within the patient cohort. This may include:
1. Identifying a patient cohort (discussed above).
2. Identifying correlative/significant titration protocol parameters.
3. Interpolating correlative/significant titration protocol parameters to determine which have the highest number of the patients successfully/unsuccessfully completing the titration protocol.
4. Adjusting the default titration protocol based on parameter values with the greatest success rates and/or fewest failures and presenting the customized titration protocol to the health care provider.

The above-described analysis may result in one or more recommended adjustments to the standard titration protocol parameters to tailor the titration protocol to a particular patient. The healthcare provider may be given the recommendation with some context. For example, the recommendation may include the patient cohort, the success rate and/or success range for the patient cohort, the initial starting dose of successful titrations, the number of days to achieve target glucose range, the recommended dosages, etc.

Depending on the desired titration output parameter, a success rate may be calculated for various personal parameters. For example, success rate=number of successful patients with personal parameter/number of patients with personal parameter.

For each significant/correlative titration protocol parameter, the health care provider may be given a recommendation to adjust the respective titration protocol parameter within the custom titration protocol based on the value from the analysis with the highest success rate.

In one non-limiting example, appropriate parameters for a patient may be selected along the following logical lines:
100 similar patients in cohort
50/100 with starting dose: 10 mg/dL=>success rate: 80%
50/100 with starting dose: 15 mg/dL=>success rate: 90%
=>choose 15 mg/dL as initial dose for this patient

Before a custom titration protocol recommendation is made, the titration system may verify that the recommendation is not contraindicated based on specific factors associated with the patient or the titration service itself. For example, a basal insulin type that may be recommended may negatively interact with another medication the patient is currently taking. Alternatively, the initial starting dose that is to be recommended could result in the patient reaching their daily insulin dose maximum prematurely. If such contraindications arise, the recommendation is either not displayed to the health care provider, or it may be displayed to the health care provider with a warning that can be overridden.

### Delivering the Titration Protocol to the Patient:

In addition to the custom titration protocol, determined as disclosed above, the titration system may also determine a preferred delivery medium for communicating the titration protocol to the patient. A similarity analysis may be conducted for demographic parameters within the similar patient cohort to determine titration success rates for the different types of delivery medium. The delivery medium with the greatest success rate for the patient's cohort may be recommended. Optimizing the titration protocol delivery medium increases the likelihood of patient compliance with the titration protocol, which increases the probability of a successful titration outcome.

For example, patients within a certain cohort might have higher success rates using a mobile application to manage their titration protocol due to the cohort members' above average use of smartphones. Whereas another cohort might be more comfortable with a less technical/featured delivery medium, such as an SMS based service that only requires the patient to respond to simple prompts.

Information about success rates with various delivery media for a given patient cohort may be along the lines of:
100 similar patients in cohort
70/100 with mobile app based service=>success rate: 90%
20/100 with SMS based service=>success rate: 70%
10/100 with smart home assistant based service=>success rate: not statistically significant
=>choose mobile app based service for this patient

The possible delivery media for the titration protocol, monitoring and data entry based on patient cohort may include: SMS, lightweight titration service app, integration in other app (e.g., mySugr), voice-skill Alexa, Siri, Cortana, Google Home (smartphone vs. home device), bot or assistant calling the patient.

### Titration System

A titration system includes a database having anonymized data for a plurality of previously managed subjects. The anonymized data may include personal parameters, e.g., demographic parameters and health parameters for each previously managed patient, the titration protocol used for each previously managed patient, and the titration output parameters for each previously managed patient.

The titration system may further include a healthcare provider interface configured to receive personal parameters for the patient. The healthcare provider interface may further be configured to receive titration output parameters to be optimized when selecting a titration protocol. Non-limiting examples of healthcare provider interfaces include personal computers, enterprise computers, dumb terminals, network communication devices, tablets, smart phones, smart watches and the like.

In some embodiments, the titration system may include a data processing device configured to generate cohorts from the plurality of subjects, identify the cohort most relevant to the patient and determine the correct antidiabetic and titration scheme for the patient.

The titration system may also include a patient user interface configured to receive administration instructions from the data processing device. Devices that may be used as a patient user interface are non-exclusive. For example, suitable patient user interfaces could include personal computers, enterprise computers, dumb terminals, television screens, bot assistants, network communication devices, tablets, smart phones, smart watches and the like.

Advantageously, titration systems and methods according to this disclosure provide customized titration protocols specifically tailored for each patient, which increases the likelihood of achieving a successful titration outcome (e.g., timely titration, minimal adverse side effects and sustained normoglycemic levels).

Embodiment 1: A method of titrating an antidiabetic for a patient, including:
a) providing a database having anonymized personal parameters for a plurality of previously managed subjects;
b) receiving patient specific personal parameters;
c) identifying, from the database having anonymized personal parameters, a patient similar cohort corresponding to the patient specific personal parameters;
d) receiving a titration output parameter to optimize for the patient; and
e) based on the titration output parameter and the patient similar cohort, deriving a customized titration protocol for the patient.

Embodiment 2: The method of embodiment 1, wherein the patient similar cohort is selected from pre-calculated cohorts generated from the plurality of previously managed subjects based on similarities in anonymized personal parameters.

Embodiment 3: The method of embodiment 1, wherein the patient similar cohort is calculated in real-time.

Embodiment 4: The method of embodiment 1, wherein the personal parameter is a health parameter that includes at least one of HbA1c, comorbid conditions, age, height, weight, body mass index, other medications, hypoglycemia risk level, blood glucose values and vital signs.

Embodiment 5: The method of embodiment 1, wherein the personal parameter is a demographic parameter that includes at least one of ethnicity, age, gender, socioeconomic status, social determinant of health and preferred mode of communication.

Embodiment 6: The method of embodiment 1, wherein the cohorts are generated using one or more of cosine similarity, knowledge graph, artificial intelligence, machine learning, clustering, k-nearest neighbor, support vector machine, naïve Bayes, Bayes network, regression, logistic regression, deep learning, neuronal network, decision tree, random forest.

Embodiment 7: The method of embodiment 1, wherein a digital twin of the patient is used to identify the patient similar cohort.

Embodiment 8: The method of embodiment 1, wherein the titration output parameter to optimize is entered by a healthcare provider.

Embodiment 9: The method of embodiment 1, wherein the titration output parameter to optimize is determined by a titration system.

Embodiment 10: The method of embodiment 1, wherein the titration output parameter to optimize is selected from the group consisting of: time to reach desired blood glucose level, percent time blood glucose level is within target range, number of hyperglycemic events, number of hypoglycemic events, titration success rate and combinations thereof.

Embodiment 11: The method of embodiment 1, wherein the customized titration protocol is based on the correlation between a titration protocol parameter and the titration output parameter to optimize.

Embodiment 12: The method of embodiment 1, wherein the customized titration protocol is determined by a regression and clustering analysis.

Embodiment 13: The method of embodiment 1, wherein step e) includes i) identifying among the plurality of previously managed subjects one or more titration protocol parameters that correlate to the titration output parameter to optimize.

Embodiment 14: The method of embodiment 13, wherein the titration protocol parameters that correlate to the titration output parameter to optimize are identified within the patient specific cohort.

Embodiment 15: The method of embodiment 13, wherein step e) further includes ii) identifying among the plurality of previously managed subjects one or more titration protocol parameters inversely correlated to the titration output parameter to optimize.

Embodiment 16: The method of embodiment 15, wherein the titration protocol parameters that inversely correlate to the titration output parameter to optimize are identified within the patient specific cohort.

Embodiment 17: The method of embodiment 15, wherein step e) further includes adjusting a default titration protocol according to the correlations identified in steps i) and ii) to derive the customized titration protocol for the patient.

Embodiment 18: The method of embodiment 1, wherein step e) includes
i) collecting a binary success variable reflecting a positive titration outcome or a negative titration outcome for the plurality of previously managed subjects;
ii) determining the titration protocol parameters with significant p-values for the positive titration outcome using a logistic regression;
iii) determining the titration protocol parameters with significant p-values for the negative titration outcome using a logistic regression;
iv) adjusting a default titration protocol according to the titration protocol parameters identified in steps ii) and iii) to derive the customized titration protocol for the patient.

Embodiment 19: The method of embodiment 18, wherein the binary success variable is collected from the patient specific cohort.

Embodiment 20: The method of embodiment 1, wherein the titration protocol includes one or more of medication type, medication subtype, initial dose of medication, dose adjustment frequency, dose adjustment increment.

Embodiment 21: The method of embodiment 1, further including:
f) determining a delivery medium most likely to result in a successful titration for the patient based on the patient cohort; and
g) delivering the titration protocol instructions to the patient using the delivery medium determined in step (f).

Embodiment 22: The method of embodiment 18, wherein the delivery medium is one or more of SMS, an application, voice-skill Alexa, Siri, Cortana, Google Home, smartphone, email, home device, bot or assistant calling the patient.

Embodiment 23: The method of embodiment 21, further including administering the antidiabetic to the patient.

Embodiment 24: The method of embodiment 23, wherein the instructions are delivered to an insulin pump and the antidiabetic is automatically administered to the patient.

Embodiment 25: A system for titrating an antidiabetic for a patient, including:
a database having anonymized personal parameters for a plurality of previously managed subjects;
a healthcare provider interface configured to receive personal parameters for the patient and to receive a titration output parameter to optimize; and
a processor configured to:
   (i) generate patient cohorts from the plurality of previously managed subjects based on commonalities in the anonymized personal parameters,
   (ii) identify from the generated cohorts a patient similar cohort corresponding to the patient specific personal parameters, and
   (iii) based on the titration output parameter and the patient similar cohort, derive a customized titration protocol for the patient.

Embodiment 26: The system of embodiment 25, wherein the health parameter includes at least one of HbA1c, comorbid conditions, age, height, weight, body mass index, other medications, hypoglycemia risk level, blood glucose values, vital signs.

Embodiment 27: The system of embodiment 25, wherein the demographic parameter includes at least one of ethnicity, age, gender, socioeconomic status, preferred mode of communication.

Embodiment 28: The system of embodiment 25, wherein the processor is configured to generate the cohorts using one or more of cosine similarity, knowledge graph, artificial intelligence, machine learning, clustering, k-nearest neighbor, support vector machine, naïve Bayes, Bayes network, regression, logistic regression, deep learning, neuronal network, decision tree, random forest.

Embodiment 29: The system of embodiment 25, wherein the processor is configured to identify the patient similar cohort using a digital twin of the patient.

Embodiment 30: The system of embodiment 25, wherein the titration output parameter to optimize is entered using the healthcare provider interface.

Embodiment 31: The system of embodiment 25, wherein the processor is configured to determine the titration output parameter to optimize.

Embodiment 32: The system of embodiment 25, wherein the titration output parameter to optimize is selected from the group consisting of: time to reach desired blood glucose level, percent time blood glucose level is within target range, number of hyperglycemic events, number of hypoglycemic events, and combinations thereof.

Embodiment 33: The system of embodiment 25, wherein the processor is configured to create the customized titration protocol based on a correlation between a titration protocol parameter and the titration output parameter to optimize.

Embodiment 34: The system of embodiment 25, wherein the processor is configured to create the customized titration protocol based on a regression and clustering analysis.

Embodiment 35: The system of embodiment 25, wherein the processor is configured to identify among the plurality of previously managed subjects one or more titration protocol parameters that correlate to the titration output parameter to optimize.

Embodiment 36: The system of embodiment 35, wherein the titration protocol parameters that correlate to the titration output parameter to optimize are identified within the patient specific cohort.

Embodiment 37: The system of embodiment 35, wherein the processor is further configured to identify among the plurality of previously managed subjects one or more titration protocol parameters inversely correlated to the titration output parameter to optimize.

Embodiment 38: The system of embodiment 37, wherein the titration protocol parameters that inversely correlate to the titration output parameter to optimize are identified within the patient specific cohort.

Embodiment 39: The system of embodiment 37, wherein the processor is further configured to adjust a default titration protocol according to the identified correlations and inverse correlations to derive the customized titration protocol for the patient.

Embodiment 40: The system of embodiment 25, wherein the processor is configured to
i) collect a binary success variable reflecting a positive titration outcome or a negative titration outcome for the plurality of previously managed subjects;
ii) determine the titration protocol parameters with significant p-values for the positive titration outcome using a logistic regression;
iii) determine the titration protocol parameters with significant p-values for the negative titration outcome using a logistic regression;
iv) adjust a default titration protocol according to the titration protocol parameters identified in steps ii) and iii) to derive the customized titration protocol for the patient.

Embodiment 41: The system of embodiment 25, wherein the binary success variable is collected from the patient specific cohort.

Embodiment 42: The system of embodiment 25, further including a patient user interface configured to receive the customized titration protocol from the data processing device.

Embodiment 43: The system of embodiment 25, wherein the processor is further configured to:
(iv) determine a delivery medium most likely to result in a successful titration for the patient based on the patient cohort, and
(v) deliver the titration protocol instructions to a patient interface.

Embodiment 44: The system of embodiment 43, wherein the delivery medium is one or more of SMS, an application, voice-skill Alexa, Siri, Cortana, Google Home, smartphone, email, home device, television, bot or assistant calling the patient.

Embodiment 45: The system of embodiment 43, further including an insulin pump, wherein the instructions are delivered to the insulin pump and the antidiabetic is automatically administered to the patient.

### BRIEF DESCRIPTION OF THE DRAWINGS

The above-mentioned aspects of exemplary embodiments will become more apparent and will be better understood by reference to the following description of the embodiments taken in conjunction with the accompanying drawings, wherein:

FIG. 1 is a schematic representation of a typical titration process;

FIG. 2 shows a schematic diagram of a custom titration system;

FIG. 3 shows a schematic diagram of a method for creating a custom titration protocol;

FIG. 4 shows a schematic diagram of a method for using a custom titration protocol according to the embodiment of FIG. 3;

FIG. 5 shows a schematic diagram of a process for placing a patient into a cohort according to the embodiments of FIGS. 3 and 4;

FIG. 6A and FIG. 6B show a schematic diagram of a method for customizing the titration protocol of a patient according to the embodiments of FIGS. 3 and 4;

FIG. 7 shows a schematic diagram of a process for communicating the titration protocol according to the embodiment of FIG. 4.

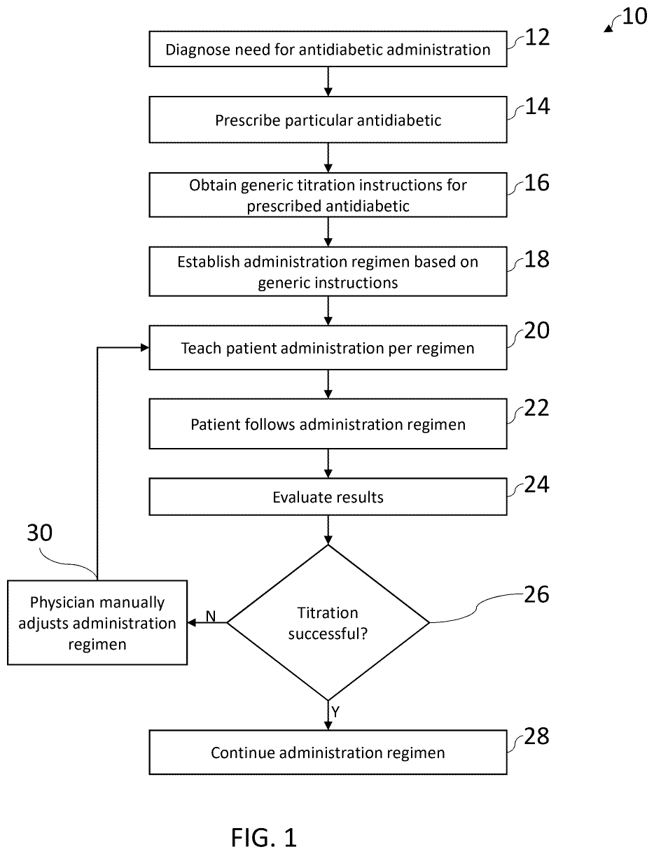

### DESCRIPTION

The embodiments described below are not intended to be exhaustive or to limit the invention to the precise forms disclosed in the following detailed description. Rather, the embodiments are chosen and described so that others skilled in the art may appreciate and understand the principles and practices of this disclosure.

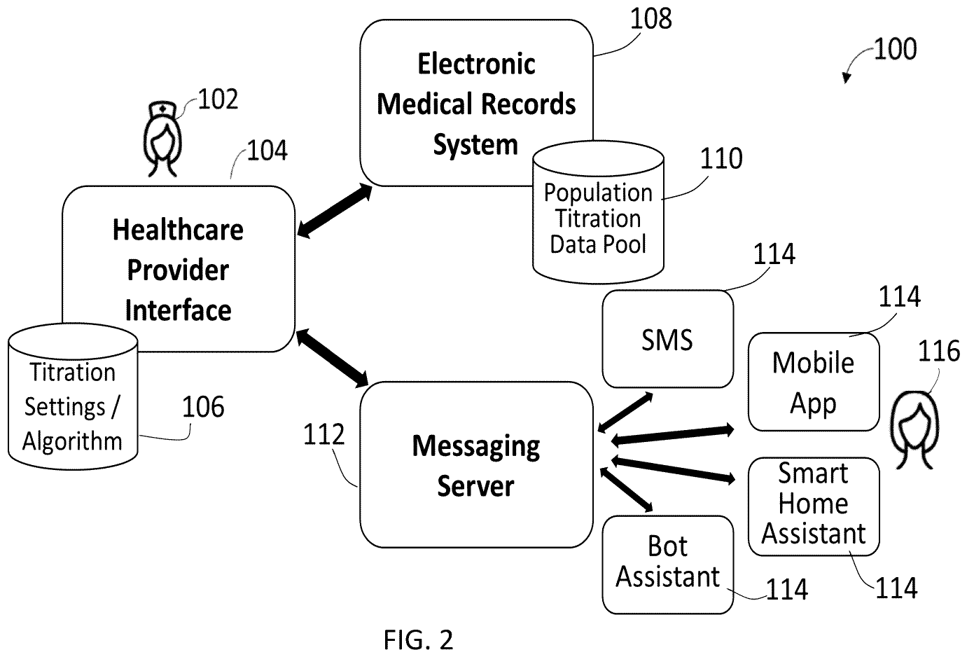

FIG. 2 is a schematic representation of a titration system 100 for customizing a titration protocol for a patient. In the embodiment shown, titration system 100 includes a healthcare provider interface 104 that allows healthcare provider 102 to interact with the titration system 100. The healthcare provider interface 104 may include both an input and an output device such as, for example, a keyboard, a mouse, a touchscreen, a display, etc. Non-limiting examples of a healthcare provider interface include personal computers, enterprise computers, dumb terminals, network communication devices, tablets, smart phones, smart watches and the like.

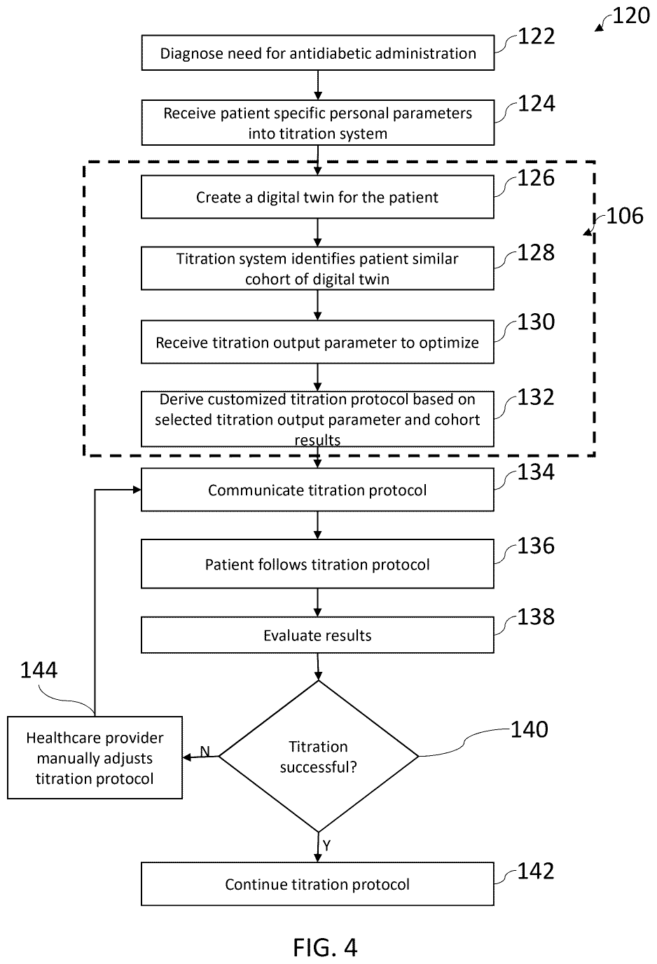

The healthcare provider interface 104 may include a processor and memory. The processor and memory may be configured to execute the method for creating a custom titration protocol 106. (Also see FIG. 4, dashed lines.) Alternatively, a separate device (not shown) having a processor and memory may be configured to execute the method for creating a custom titration protocol 106. In embodiments having such a separate device, the separate device may be configured to communicate with the healthcare provider interface and perform some or all of the functions of the healthcare provider interface described herein.

A population titration data pool 110 may be housed in an electronic medical records system 108. The healthcare provider interface 104 may be connected to the electronic medical records system 108 using wired or wireless communication technology.

The healthcare provider interface 104 may also be connected to a messaging server 112. The messaging server 112 may be configured to communicate instructions for the titration protocol to the patient 116 using one or more delivery media 114. For example, instructions may be sent to patient 116 using SMS, a mobile app, a smart home assistant, a bot assistant, any other suitable delivery medium or combinations thereof.

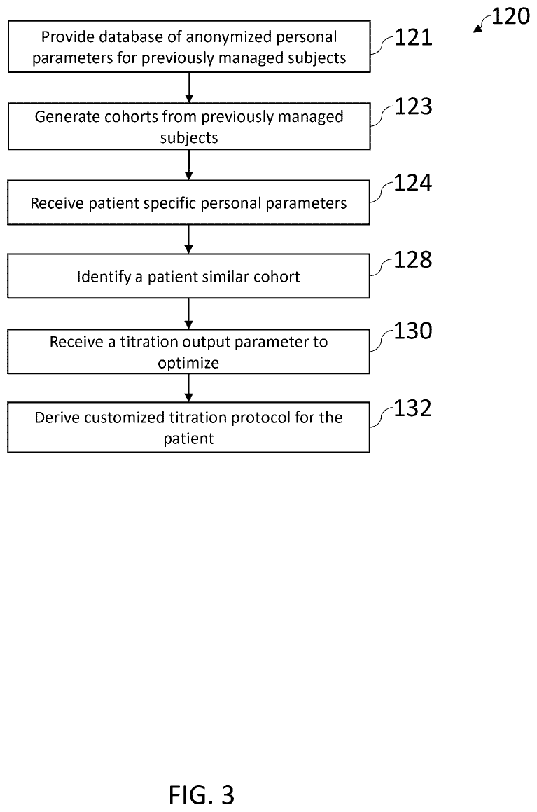

FIG. 3 shows a block diagram of a non-limiting embodiment of a method for creating a custom titration protocol 120 according to this disclosure. The method steps disclosed herein can be carried out in the illustrated sequence. However, alternative sequences are also possible. Further, individual or multiple method steps can be carried out in parallel, simultaneously, or repeatedly, either on their own or in groups. For example, the steps for determining titration protocol parameters need not be carried out in the precise order described below. Furthermore, the method can comprise additional method steps that are not illustrated. Independently of the fact that the term method step is used, the term “step” says nothing about the duration of the method steps. Thus, the specified method steps can, individually or in groups, be carried out briefly, but can also be carried out over a longer time period, for example, over time intervals of a number of minutes, hours, days, weeks or even months, for example, continuously or repeatedly.

Initially, at step 121, a database 110 of anonymized personal parameters for previously managed subjects (also referred to herein as a population titration data pool) is provided to the titration system. The database 110 may be obtained from, for example, electronic medical records.

In step 123, a similarity analysis of the anonymized personal parameters, e.g., the health and demographic parameters for the previously managed subjects may be performed. The previously managed subjects may then be grouped into cohorts based on the results of the similarity analysis. As discussed above, non-limiting examples of similarity analysis techniques suitable for use in step 123 include cosine similarity, knowledge graph, artificial intelligence, machine learning, clustering, k-nearest neighbor, support vector machine, naïve Bayes, Bayes network, regression, logistic regression, deep learning, neuronal network, decision tree, random forest.

At step 124, the titration system receives the patient specific personal parameters. For example, the healthcare provider 102 may enter the patient's health and demographic parameters into the titration system 100. This may be accomplished using the healthcare provider interface 104. Alternatively, the titration system may access this information directly from the patient's electronic medical records.

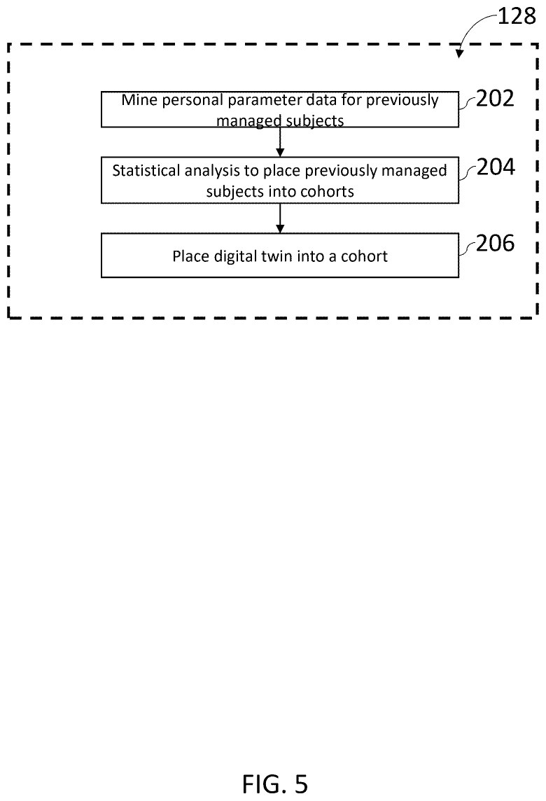

A patient similar cohort is identified at step 128. This may be accomplished by performing a similarity analysis between the patient and the cohorts of previously managed subjects using techniques such as cosine similarity, knowledge graph, artificial intelligence, machine learning, clustering, k-nearest neighbor, support vector machine, naïve Bayes, Bayes network, regression, logistic regression, deep learning, neuronal network, decision tree, random forest. This step is discussed in more detail with reference to FIGS. 4 and 5, below.

At step 130, a titration output parameter to optimize is received by the titration system. The titration output parameter to optimize may be entered by a healthcare provider 102 using the healthcare provider interface 104. In the alternative, the titration output parameter to optimize may be derived based on a statistical analysis of the patient specific cohort.

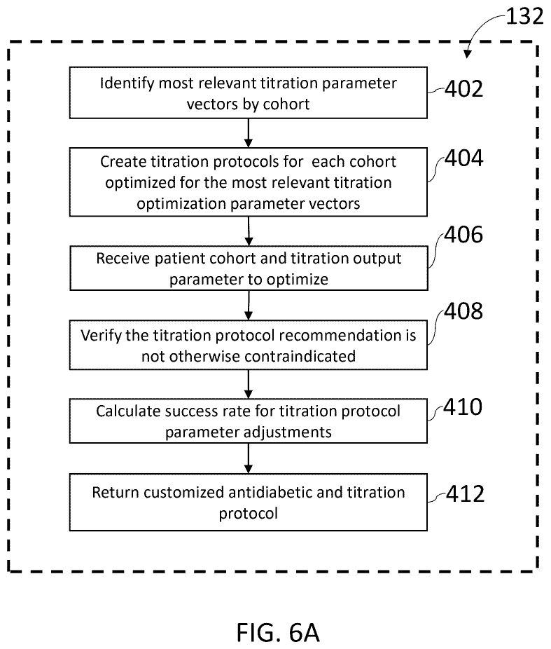

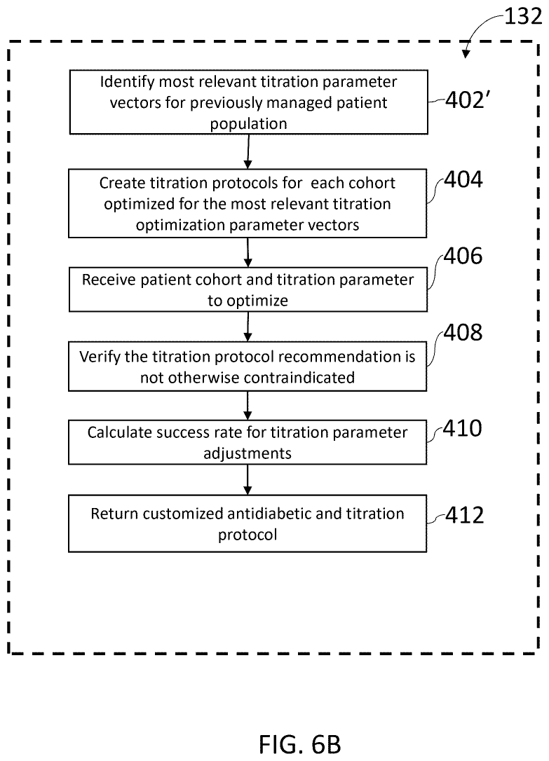

The titration system may then derive a customized titration protocol for the patient at step 132. The customized titration protocol may be based on the results of previously managed subjects in the patient specific cohort. It may also take into account any contraindications particular to the patient. Alternative methods for creating the customized titration protocol are discussed in more detail with reference to FIGS. 4, 6A and 6B, below.

FIG. 4 shows a block diagram of a non-limiting embodiment of the method for using a custom titration protocol to treat a patient. The embodiment depicted in FIG. 4 uses the same methodology for creating the custom titration protocol as shown in FIG. 3 and like steps have like numbers. The method shown in FIG. 4 may also be implemented using the titration system 100 shown in FIG. 2.

In step 122, a healthcare provider diagnoses the need for an antidiabetic. This may be accomplished either during a regularly scheduled office visit or by an examination performed in response to patient symptoms such as frequent urination, thirst, unexplained weight loss or symptoms of more severe complications of diabetes or comorbid conditions.

At step 124, the titration system receives the patient specific personal parameters. For example, the healthcare provider 102 may enter the patient's health and demographic parameters into the titration system 100. This may be accomplished using the healthcare provider interface 104. Alternatively, the titration system may access this information directly from the patient's electronic medical records.

In step 126, the patient's personal parameters may be used to create a digital twin. Alternatively, the digital twin may be selected from an existing set of common profiles. The common profile that most closely parallels the patient's data can be selected and used as the digital twin. The digital twin need not be perfectly identical to the patient for purposes of this disclosure. It need only represent the patient's health and demographic parameters sufficiently to perform the disclosed statistical analysis.

In step 128, shown in more detail in FIG. 5, the titration system identifies a patient cohort corresponding to the digital twin. As shown in FIG. 5, data regarding previously managed subjects may be mined in step 202 from the electronic medical records system 108. In particular, the data may include anonymized personal parameters, such as health and demographic parameters for previously managed subjects.

In step 204, a similarity analysis of the anonymized personal parameters for the previously managed subjects may be performed. The previously managed subjects may then be grouped into cohorts based on the results of the similarity analysis. As discussed above, non-limiting examples of similarity analysis techniques suitable for use in step 204 include cosine similarity, knowledge graph, artificial intelligence, machine learning, clustering, k-nearest neighbor, support vector machine, naïve Bayes, Bayes network, regression, logistic regression, deep learning, neuronal network, decision tree, random forest.

Finally, at step 206, the digital twin may be placed into a cohort with the previously managed subjects. This may be accomplished using similarity analysis. Several examples of similarity analysis techniques suitable for use with this disclosure are discussed elsewhere herein.

Returning to FIG. 4, at step 130, one or more titration output parameters may be selected for optimization in the custom titration protocol. In one non-limiting embodiment, the healthcare provider may select the titration output parameters to optimize and enter them into the titration management system using the healthcare provider interface 104. Other methods of selecting and entering the titration parameters are also contemplated. For example, a set of parameters to optimize may be derived by the titration system based on a statistical analysis of the patient similar cohort.

At step 132, shown in more detail in FIGS. 6A and 6B, the titration system optimizes the titration protocol based on the selected titration output parameter and results from the previously managed patient cohort to which the digital twin has been assigned.

In one alternative embodiment, shown in FIG. 6A, at step 402, data from previous patients within each cohort with titrations satisfying the optimization goals or failing to satisfy the titration output optimization goals can be isolated and correlative protocol parameters may be determined. For example, each of the titration protocol parameters could be assessed in terms of their statistical correlation to the desired/undesired titration outcomes vs. all possible outcomes. Within acceptable confidence and significance levels, titration parameters at or above a minimum statistical correlation threshold (e.g., 0.7, 0.8, 0.9 or 0.95) could be considered for adjustment in the custom titration protocol.

Alternatively, at step 402, a regression and clustering analysis to compare the parameters of a successful titration could be used to identify which characteristics were deterministic of success for the particular cohort of previously managed subjects. At the same time, a regression and clustering analysis to compare the parameters of an unsuccessful titration could be used to identify which characteristics were deterministic of failure. A logistic regression approach may include, for example:
1. Collecting patient health and demographic parameters, including a success variable, which may be, e.g., binary.
2. Using the success variable as the response/target variable.
3. Initially assuming all protocol parameters are predictors for the regression.
4. Identifying parameters with a significant p-value, meaning that such parameters have a significant effect on the outcome and are to some extent deterministic of success or failure.
5. Carrying the parameters identified as meeting the significance criteria to step 404.

It is also possible to perform the above-described analysis on the entire previously managed patient pool 110, as shown in FIG. 6B.

In the alternative embodiment shown in FIG. 6B, at step 402′, data from previously managed subjects with titrations satisfying the optimization goals or failing to satisfy the titration output optimization goals can be isolated and correlative protocol parameters may be determined. For example, each of the titration protocol parameters could be assessed in terms of their statistical correlation to the desired/undesired titration outcomes vs. all possible outcomes. Within acceptable confidence and significance levels, titration parameters at or above a minimum statistical correlation threshold (e.g., 0.7, 0.8, 0.9 or 0.95) could be considered for adjustment in the custom titration protocol.

Alternatively, at step 402′, a regression and clustering analysis to compare the parameters of a successful titration could be used to identify which characteristics were deterministic of success. At the same time, a regression and clustering analysis to compare the parameters of an unsuccessful titration could be used to identify which characteristics were deterministic of failure. A logistic regression approach may include, for example:
1. Collecting patient health and demographic parameters, including a success variable, which may be, e.g., binary.
2. Using the success variable as the response/target variable.
3. Initially assuming all protocol parameters are predictors for the regression.
4. Identifying parameters with a significant p-value, meaning that such parameters have a significant effect on the outcome and are to some extent deterministic of success or failure.
5. Carrying the parameters identified as meeting the significance criteria to step 404.

At step 404, a set of titration protocols for different optimization parameters may be created for each cohort. This can be done by:
a. Interpolating correlative/significant titration parameters to determine which titration protocol values have the highest number of the patients successfully completing the titration protocol.
b. Adjusting the default titration protocol based on titration protocol values with the greatest success rates.

At step 406, the titration system may receive the patient cohort and a titration output parameter to optimize. In one example, the titration parameter to optimize may be entered by the health care provider 102 using the healthcare provider interface 104. In another embodiment the patient cohort and/or the titration parameter to optimize may be derived based on data from previously managed subjects.

As shown in FIGS. 6A and 6B, at step 408, before a custom titration protocol recommendation is made, the titration system may verify that the recommendation is not contraindicated based on specific factors associated with the patient or the titration service itself. For example, a basal insulin type that may be recommended may negatively interact with another medication the patient is currently taking. Alternatively, the initial starting dose that is to be recommended could result in the patient reaching their daily insulin dose maximum prematurely. If such contraindications arise, the recommendation is either not displayed to the health care provider, or it may be displayed to the health care provider with a warning that can be overridden.

The above-described analysis may result in one or more recommended adjustments to the standard titration protocol parameters to tailor the titration protocol to a particular patient. At step 410, a success rate for the titration protocol parameter adjustments may be calculated for various personal parameters. For example, success rate=number of successful patients with personal parameter/number of patients with personal parameter.

Finally, at step 412, the titration system returns a customized antidiabetic and titration protocol based on the patient's cohort and the titration output parameters selected for optimization.

Returning to FIG. 4, once the customized titration protocol has been returned, it must be communicated at step 134.

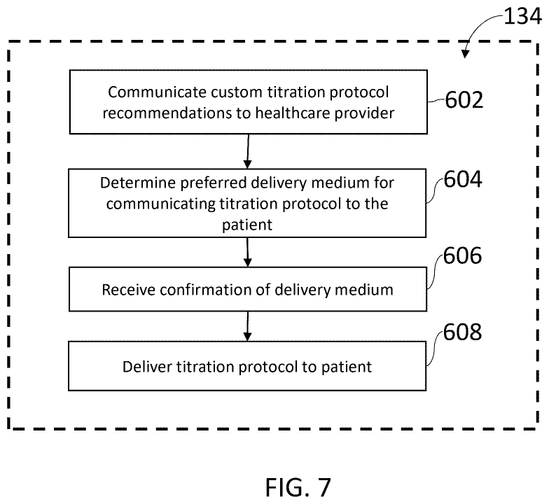

FIG. 7 shows the process for communicating the titration protocol in more detail. At step 602, the health care provider may be given a recommendation to adjust each significant/correlative titration protocol parameter within the custom titration protocol based on the value from the step 410 analysis with the highest success rate. The healthcare provider may be given the titration protocol recommendation at 602 with some context. For example, the recommendation may include the patient cohort, the success rate and/or success range for the patient cohort, the initial starting dose of successful titrations, the number of days to achieve target glucose range, the recommended dosages, etc.

In one non-limiting example, appropriate parameters for a patient may be communicated along the following logical lines:
100 similar patients in cohort
50/100 with starting dose: 10 mg/dL=>success rate: 80%
50/100 with starting dose: 15 mg/dL=>success rate: 90%
=>choose 15 mg/dL as initial dose for this patient

At step 604, the titration system may also determine a preferred delivery medium for communicating the titration protocol to the patient. A similarity analysis may be conducted for demographic parameters within the similar patient cohort to determine titration success rates for the different types of delivery media. The delivery medium with the greatest success rate for the patient's cohort may be recommended via the healthcare provider interface 104. Optimizing the titration protocol delivery medium increases the likelihood of patient compliance during the protocol, which increases the probability of a successful titration outcome.

For example, patients within a certain cohort might have higher success rates using a mobile application to manage their titration protocol due to the cohorts' above average use of smartphones. Whereas another cohort might be more comfortable with a less technical/featured delivery medium, such as an SMS based service that only requires the patient to respond to simple prompts.

Information about success rates with various delivery media for a given patient cohort may be along the lines of:
100 similar patients in cohort
70/100 with mobile app based service=>success rate: 90%
20/100 with SMS based service=>success rate: 70%
10/100 with smart home assistant based service=>success rate: not statistically significant
=>choose mobile app based service for this patient

The possible delivery media for the titration protocol, monitoring and data entry based on patient cohort may include: SMS, lightweight titration service app, integration in other app (e.g., mySugr), voice-skill Alexa, Siri, Cortana, Google Home (smartphone vs. home device), bot or assistant calling the patient. Since there is always the possibility that a particular patient does not have access to the preferred delivery medium for his or her cohort, the healthcare provider and/or the patient may optionally confirm the selection of the delivery medium at step 606.

At step 608, the custom titration protocol is delivered to the patient and, returning to FIG. 4, the patient follows the administration regimen at step 136. Results, such as fasting blood glucose, hyperglycemic and hypoglycemic events, time in range, etc. can then be evaluated at step 138. If the titration is successful at step 140, then the patient may continue using the protocol at step 142. If the titration is not successful at step 140, then the healthcare provider may manually adjust the titration protocol at step 144.

While exemplary embodiments have been disclosed hereinabove, the present invention is not limited to the disclosed embodiments. Instead, this application is intended to cover any variations, uses, or adaptations of this disclosure using its general principles. Further, this application is intended to cover such departures from the present disclosure as come within known or customary practice in the art to which this invention pertains and which fall within the limits of the appended claims.

What is claimed is:

1. A method of titrating an antidiabetic for a patient, comprising:
a) providing a database having anonymized personal parameters for a plurality of previously managed subjects;
b) receiving patient specific personal parameters;
c) identifying, from the database having anonymized personal parameters, a patient similar cohort corresponding to the patient specific personal parameters;
d) receiving a titration output parameter to optimize for the patient; and
e) based on the titration output parameter and the patient similar cohort, deriving a customized titration protocol for the patient.

2. The method of claim 1, wherein the patient similar cohort is selected from pre-calculated cohorts generated from the plurality of previously managed subjects based on similarities in anonymized personal parameters.

3. The method of claim 1, wherein the patient similar cohort is calculated in real-time.

4. The method of claim 1, wherein the cohorts are generated using one or more of cosine similarity, knowledge graph, artificial intelligence, machine learning, clustering, k-nearest neighbor, support vector machine, naïve Bayes, Bayes network, regression, logistic regression, deep learning, neuronal network, decision tree, random forest.

5. The method of claim 1, wherein a digital twin of the patient is used to identify the patient similar cohort.

6. The method of claim 1, wherein the titration output parameter to optimize is selected from the group consisting of: time to reach desired blood glucose level, percent time blood glucose level is within target range, number of hyperglycemic events, number of hypoglycemic events, titration success rate and combinations thereof.

7. The method of claim 1, wherein step e) comprises i) identifying among the plurality of previously managed subjects one or more titration protocol parameters that correlate to the titration output parameter to optimize.

8. The method of claim 7, wherein step e) further comprises ii) identifying among the plurality of previously managed subjects one or more titration protocol parameters inversely correlated to the titration output parameter to optimize.

9. The method of claim 8, wherein step e) further comprises adjusting a default titration protocol according to the correlations identified in steps i) and ii) to derive the customized titration protocol for the patient.

10. The method of claim 1, wherein step e) comprises
i) collecting a binary success variable reflecting a positive titration outcome or a negative titration outcome for the plurality of previously managed subjects;
ii) determining the titration protocol parameters with significant p-values for the positive titration outcome using a logistic regression;
iii) determining the titration protocol parameters with significant p-values for the negative titration outcome using a logistic regression;
iv) adjusting a default titration protocol according to the titration protocol parameters identified in steps ii) and iii) to derive the customized titration protocol for the patient.

11. The method of claim 10, wherein the binary success variable is collected from the patient specific cohort.

12. The method of claim 1, further comprising:
f) determining a delivery medium most likely to result in a successful titration for the patient based on the patient cohort; and
g) delivering the titration protocol instructions to the patient using the delivery medium determined in step (f).

13. The method of claim 12, wherein the instructions are delivered to an insulin pump and the antidiabetic is automatically administered to the patient.

14. A system for titrating an antidiabetic for a patient, comprising:
a database having anonymized personal parameters for a plurality of previously managed subjects;
a healthcare provider interface configured to receive personal parameters for the patient and to receive a titration output parameter to optimize; and
a processor configured to:
(i) generate patient cohorts from the plurality of previously managed subjects based on commonalities in the anonymized personal parameters,
(ii) identify from the generated cohorts a patient similar cohort corresponding to the patient specific personal parameters, and
(iii) based on the titration output parameter and the patient similar cohort, derive a customized titration protocol for the patient.

15. The system of claim 14, wherein the processor is configured to generate the cohorts using one or more of cosine similarity, knowledge graph, artificial intelligence, machine learning, clustering, k-nearest neighbor, support vector machine, naïve Bayes, Bayes network, regression, logistic regression, deep learning, neuronal network, decision tree, random forest.

16. The system of claim 14, wherein the processor is configured to identify the patient similar cohort using a digital twin of the patient.

17. The system of claim 14, wherein the titration output parameter to optimize is selected from the group consisting of: time to reach desired blood glucose level, percent time blood glucose level is within target range, number of hyperglycemic events, number of hypoglycemic events, and combinations thereof.

18. The system of claim 20, wherein the processor is configured to create the customized titration protocol based on a correlation between a titration protocol parameter and the titration output parameter to optimize.

19. The system of claim 14, wherein the processor is configured to identify among the plurality of previously managed subjects one or more titration protocol parameters that correlate to the titration output parameter to optimize.

20. The system of claim 19, wherein the processor is further configured to identify among the plurality of previously managed subjects one or more titration protocol parameters inversely correlated to the titration output parameter to optimize.

21. The system of claim 20, wherein the processor is further configured to adjust a default titration protocol according to the identified correlations and inverse correlations to derive the customized titration protocol for the patient.

22. The system of claim 14, wherein the processor is configured to
i) collect a binary success variable reflecting a positive titration outcome or a negative titration outcome for the plurality of previously managed subjects;
ii) determine the titration protocol parameters with significant p-values for the positive titration outcome using a logistic regression;
iii) determine the titration protocol parameters with significant p-values for the negative titration outcome using a logistic regression;
iv) adjust a default titration protocol according to the titration protocol parameters identified in steps ii) and iii) to derive the customized titration protocol for the patient.

23. The system of claim 22, wherein the binary success variable is collected from the patient specific cohort.

24. The system of claim 43, further comprising an insulin pump, wherein the instructions are delivered to the insulin pump and the antidiabetic is automatically administered to the patient.
